(19) United States
(12) Patent Application Publication
(10) Pub. No.: US 2020/0285771 A1
(43) Pub. Date: Sep. 10, 2020

(54) SYSTEM AND METHOD FOR REMOVING PERSONALLY IDENTIFIABLE INFORMATION FROM MEDICAL DATA

(71) Applicants: Abhishek Dey, Guahati (IN); Sam Tkach, Brooklyn, NY (US); Mark Ruiz, San Francisco, CA (US); Ray Rahman, San Francisco, CA (US)

(72) Inventors: Abhishek Dey, Guahati (IN); Sam Tkach, Brooklyn, NY (US); Mark Ruiz, San Francisco, CA (US); Ray Rahman, San Francisco, CA (US)

(21) Appl. No.: 16/292,519

(22) Filed: Mar. 5, 2019

(51) Int. Cl. G06F 21/62; G16H 30/20

(52) U.S. Cl. CPC G06F 21/6254; G06Q 10/0635; G16H 30/20; G06F 21/6272

(57) ABSTRACT

A system and method for removing personally identifiable information (PII) from medical data and ensuring strict compliance with privacy laws and regulations. The PII removal tool removes, deletes or redacts sensitive personally identifiable information, which may comprise any health care data that may be linked to any individual. The system and method is preferably applied to medical data, images, video, or other electronic media captured from medical procedures, endoscopes, or arthroscopic surgery. Artificial intelligence powered software and models are applied to the medical data for the detection of PII comprising patient names, medical history, faces, account numbers, digital identities, biometric data, or other sensitive personal information which may be linked to an individual. Medical data is re-written without PII and the system generates metadata describing the deleted PII. Privacy law compliant medical data is normalized, stored hierarchically, and transmitted to third party storage systems for analysis with software models for medical insights, anatomical recognition, surgical metrics, patient outcome, diagnosis and medical payment and billing.

### BACKGROUND

There are potentially large amounts of data available from endoscopic surgeries and medical procedures in which a doctor or surgeon utilizes an optical device with a lens and camera to capture medical images and video and look inside the human body. Endoscopes are an illuminated optical instrument used to look inside the body and are typically used to examine the stomach, intestine, rectum, nose, bronchial tube, ear, or urinary tract, etc. Arthroscopes are a type of endoscope that is inserted into a joint through a small incision, such as during knee surgery, ACL reconstruction, diagnosis and treatment of hip joints, shoulder problems, rotator cuff tears, wrist injuries and pains, or spinal procedures.

During endoscopic surgeries, multiple portals or incisions are made in the body for the insertion of the arthroscope or surgical tools. The camera typically continues to record while it is adjusted and re-inserted into the body, and may from time to time, record and pick up personally identifiable information (PII) from people and faces outside the body in the operating room. The video data from the surgery may also contain personally identifiable information such as the patient's name, medical history, or other protected health information. The HIPAA Privacy Rule is a federal regulation which governs the use and disclosure of protected health information, which is any information regarding health status, health care, or medical history, which can be linked to any individual. The privacy rule and regulations obligate medical service providers and other entities to make certain legally required disclosures, notifications to the individual, and documentation of privacy policies.

In order to comply with privacy laws and regulations, there is a need to safely remove and delete personally identifiable information from medical imaging and video data generated from endoscopic surgeries and procedures. With the removal of personally identifiable information and strict compliance with privacy laws, patient anonymous data may be used to build labeled data sets of diagnosed injuries, surgical metadata, and other metrics.

### SUMMARY

A preferred embodiment of the presently described invention may be a computer hardware and software system and method for removing personally identifiable information (PII) from medical imaging video data. The medical imaging video may preferably be obtained from endoscopic surgery cameras and procedures. Personally identifiable information to be removed from the video data may include the patient's name, medical history, or any information regarding health care which can be linked to any individual, or patient. Additionally, any other personally identifiable information which may be linked to any doctors, surgeons, nurses and health care providers may also be removed with the presently described computer hardware and software system and method.

The presently described system and method for removing personally identifiable information provides for strict compliance with healthcare data privacy laws and regulations. By removing any personally identifiable information from the medical imaging video data, the resulting cleansed data may enter a secure data ingestion pipeline and be transmitted, uploaded, or downloaded to various third parties for processing with additional software algorithms, systems and methods. Massive amounts of medical imaging video data from endoscopic surgeries may be processed with the presently described system and method for building a complimentary massive data set of privacy law compliant medical data and metadata. Artificial intelligence (AI) powered software, models and filters may preferably be used to detect and remove any personally identifiable information in the video and medical data.

A preferred embodiment of the present invention may be a data ingestion service that is rolled out and integrated with existing health care computer and information technology systems, and made available to doctors, surgeons and other health care providers. During endoscopic surgeries and procedures, one or more cameras or imaging devices may record the medical procedure onto a digital media device such as a computer hard drive, flash drive or other non-volatile media. While in the doctor's possession, the media device may contain personally identifiable information, such as the patient's name or medical history. The presently described data ingestion service may be embodied in a hardware and software tool set which may interface with the health care provider's computer systems and apply artificial intelligence (AI) powered software to read the surgical procedure video data into memory. In run-time, the AI models may preferably detect any potential personally identifiable information (PII) and pro-actively remove or delete the PII information from the video. The data ingestion service may preferably re-write onto another media device, or transfer to a cloud based data storage service, the cleansed video data along with metadata that describes, at an anonymous level, the removed PII. In a preferred embodiment, at no point during data ingestion does the system and method write PII onto the computer systems of the health care provider, or transfer any PII onto a secondary media device or cloud based storage system.

In another preferred embodiment of the system and method, a media device containing surgical video data is connected to the data ingestion service. The data may be encrypted and logged on a safe transport device or data upload connection. In the ingestion pipeline, a secondary tool set may be applied to the video data to detect and remove any other personally identifiable information present in the video or medical imaging data. The PII removal, deletion and cleansing is preferably provided in a AI-powered framework with multiple artificial intelligence models, depending on the PII information that is detected and classified in the use case, and which may be dependent on the device type, manufacturer, and other patient data encoded in the video frames. The data may be preferably uploaded onto a medical video storage device, or a cloud based repository, and run through normalization processes, and stored in a hierarchical structure.

In a preferred embodiment, the data ingestion service provides an output of medical video data from endoscopic surgeries that is free from any detectable PII, and absolutely no health care information that can be linked to any specific individual. By preferably applying the data ingestion service to a large data set of medical video data, the service may preferably build a large data set of medical video for processing with AI-powered tools for computing anatomical and clinical diagnosis, classification, recognition and identification methods. The data set may be used to find and detect specific anatomical features, bones, ligaments, and diagnosis of disease or injuries.

In a preferred embodiment of the system and method, the medical video data may be organized into individual frames and processed on a frame by frame basis for identifying different activity from the frames, such as periods of detectable PII. The video data may be further edited and reconstructed without the frames and periods of detectable PII. Metadata may preferably be generated by the system and method to describe any segments of the video with detectable PII. Additionally, the system and method may be extended to understanding other media types, text documents, or images, and alternative AI-powered models may be used to remove different types of personally identifiable information, such as patient names, medical history, social security numbers, bank accounts, billing information, home addresses, phone numbers, and dates and times.

### DESCRIPTION OF THE DRAWINGS

FIG. 1 is a system diagram of the system and method for removing personally identifiable information from medical data; in a preferred cloud based service architecture.

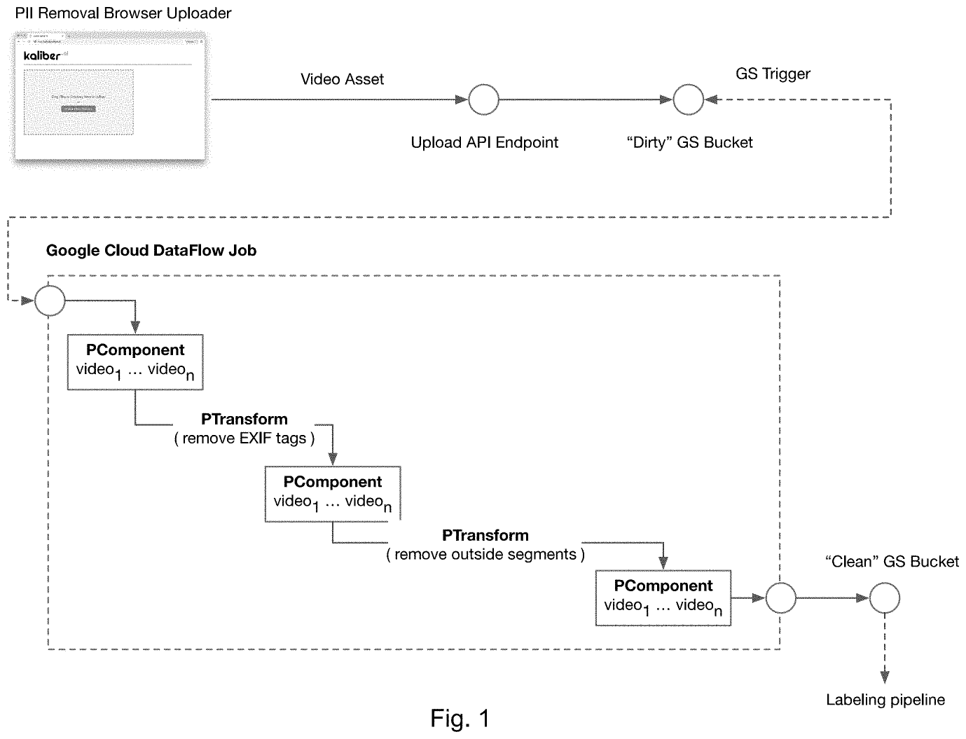

FIG. 2 is a browser based uploader or client application for selecting medical data files to be uploaded to the system and method for removing personally identifiable information from medical data.

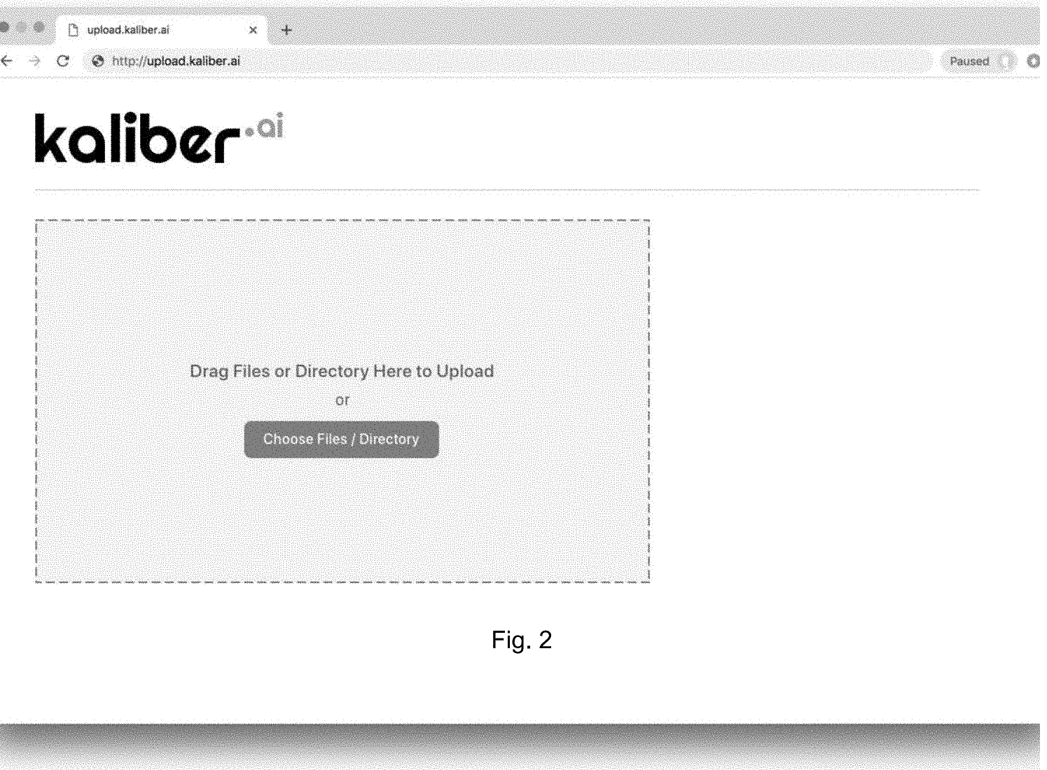

FIG. 3 is a browser based uploader or client application for selecting medical data files to be uploaded to the system and method for removing personally identifiable information from medical data; here the patient name(s), patient.txt, medical video file(s), and medical image(s), are provided in the user interface, along with an upload button.

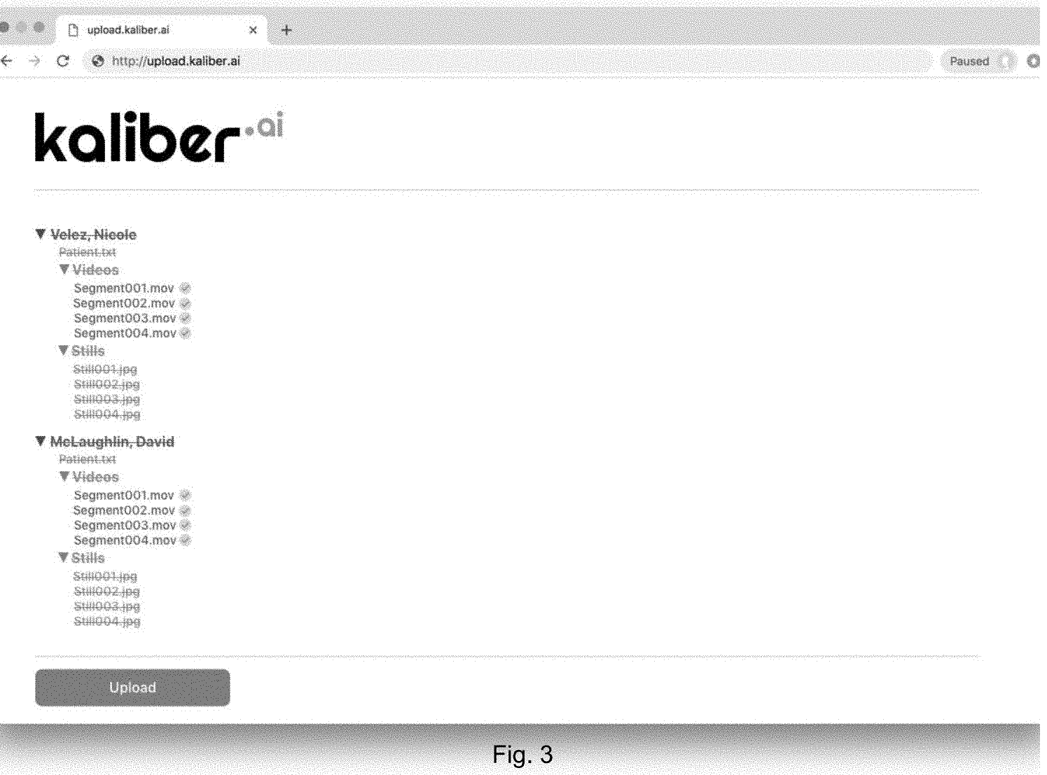

FIG. 4 is a browser based uploader or client application for selecting and uploading medical data files into the system and method for removing personally identifiable information from medical data; here the uploading status of the files is shown.

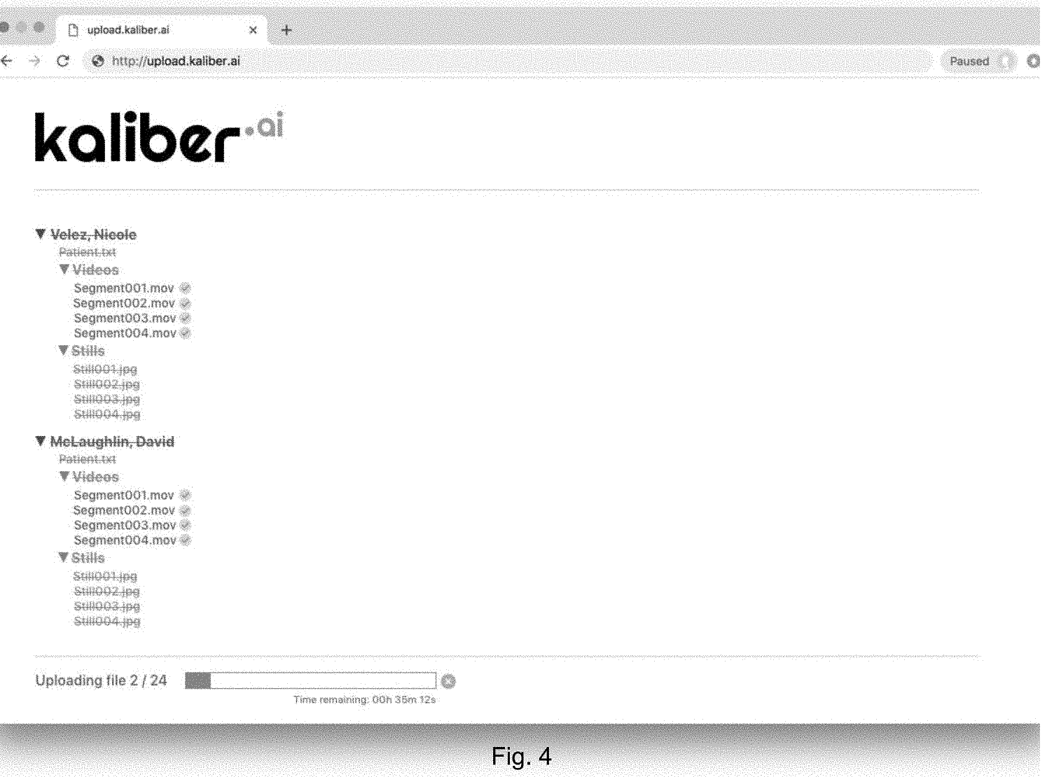

FIG. 5 is a standalone native application and user interface for selecting medical data for removal of personally identifiable information; a source drive is chosen, and a destination drive is chosen for the output and encryption of medical data that has been scrubbed or removed of any personally identifiable information.

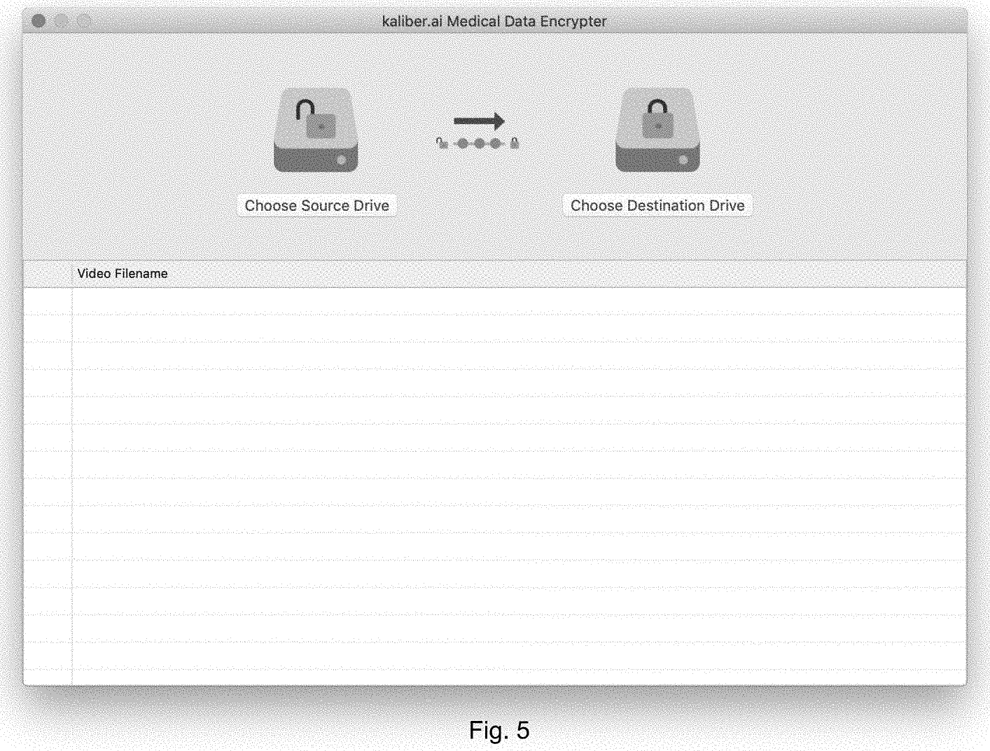

### DETAILED DESCRIPTION

In a preferred embodiment of the system and method, electronic medical and clinical data is captured, recorded, and stored from medical procedures, surgeries, examinations, and other health care provider patient interactions. Medical imaging media may preferably be comprised of images and video from endoscopic procedures and surgeries, arthroscopic surgeries, or any other data captured with an optical device with a lens and camera used to look inside the human body. Imaging devices are typically used by doctors, surgeons, or other medical professionals during medical procedures. The medical imaging data, video, images, and other electronically captured signals and data is typically stored and recorded onto a non-volatile media storage device, such as a computer hard drive, or flash drive, which is interfaced with the imaging device. The imaging device, digital camera, endoscopes or arthroscope is ordinarily kept in the operating room and organized with other surgical or medical devices, and sterilized with typical autoclavable methods. However, the media storage device may be kept with the doctor or medical professional and taken out of the operating room for viewing, downloading and processing the medical imaging data.

In a preferred embodiment of the system and method, the medical imaging data is interfaced with a data ingestion pipeline and service. From here, the portable media storage device is connected to the health care provider's computer and information technology systems. The medical imaging data may preferably be encrypted, logged and securely stored on the health care providers computer systems. A secondary software and hardware toolset may preferably be applied to the medical imaging data for uploading, transmitting, or downloading the data to a cloud based repository or storage system, which is interfaced with the health care providers information technology systems.

In a preferred embodiment of the system and method, the medical data is received from health care providers with a web browser based tool and website; which the provider may connect to and load on a client device, and upload a selection of medical data files. A preferred embodiment of the website and browser bade tool may be a drag and drop user interface, for selecting and uploading medical data files to the service. The browser based tool and website may automatically detect text files, images, and other medical data files with personally identifiable information (PII), and discard the PII from being uploaded. For example, in a preferred embodiment, the medical images and videos may contain the patient's name attached to the file folder name. The system will rename the folder or files containing the patient's name in the filename, in order to discard, delete, and remove the PII. The system additionally providers a confirmation to the health care provider that the PII has been removed and that the filenames or folder names are being renamed, to not contain PII.

In a preferred embodiment of the system and method, additional processing is applied to remove any embedded personally identifiable information (PII). The embedded PII may potentially contain metadata within the medical data files. The system preferably uses a multi-pass approach to removed embedded personally identifiable information tags in the medical data files. Specific artificial intelligence (AI) models may preferably detect, for example, when the medical images or videos of a surgery, are outside the body and recording personally identifiable information (PII), such as people's faces. The system AI models mark these segments containing PII, remove them from the medical video or image files, and then re-assemble the medical data files, images and video, and outputs the data for the next processing stage.

In a preferred embodiment of the system and method, artificial intelligence (AI) powered software and image recognition algorithms, and models are applied to the medical imaging data to detect personally identifiable information (PII). The AI models are applied to find and detect any instances of personally identifiable information, which may preferably include any health care information or medical history, which can be linked to any individual, and additionally, patient names, medical professional names, faces, home addresses, email addresses, telephone numbers, social security numbers, account numbers, digital identities, passwords, login information, screen names, dates of birth, age, genetic information, or biometric data. The medical imaging data may be deconstructed into individual frames for detecting PII on a frame by frame basis, and preferably the system may label, mark, tag, or code the beginning and ending of segments of video data frames in which PII is present. In a preferred application of the system and method AI models, the medical imaging data will be labeled and coded on a frame-level basis for the presence of PII.

In a preferred embodiment of the system and method, all personally identifiable information (PII) is removed, deleted, erased, censored, bleeped, cut, expunged or redacted from the medical imaging data. PII present in medical imaging video may be deleted on a frame by frame basis, which may include video frames, image stills, image segments, as well as audio data. In the instances of medical images, PII may be removed by deleting entire images, or redacting portions of images. For other types of electronic medical data and signals, PII may be deleted by reducing the data to randomized noise or static. In a preferred embodiment, the medical imaging data containing PII is marked for deletion and immediately overwritten by the health care provider's computer and information technology system or local operating system.

In a preferred embodiment of the system and method, the data ingestion service outputs and re-writes the medical imaging data without PII and generates metadata, or reporting metrics, describing the deleted PII. From the ingested medical imaging data, the system and method preferably generates medical imaging data, without PII, as extracted from the initially provided raw medical imaging data. Metadata describing the deleted and removed personally identifiable information may additionally be generated by the system, reporting on the amount, classification types, frame-level metrics, and other statistics regarding the PII. However, the PII metadata generated by the system will preferably not expose any personal information which may be linked to any individual.

In a preferred embodiment of the system and method, the output medical imaging data may be transmitted, uploaded or downloaded to secondary data storage systems on the health care provider's computer and information technology systems or made available to third party information services and storage systems. The cleansed medical imaging data is then preferably normalized to application specific standards, and stored in a hierarchical structure to make available for additional secondary processing methods. The medical imaging data is preferably without any personally identifiable information (PII) at this stage in the data ingestion and collection process. Third party services may access and download, or otherwise receive, the PII compliant medical imaging data at this stage and further apply application specific data processing and manipulation.

In a preferred embodiment of the system and method, strict compliance with privacy laws and regulations is ensured with AI-powered models, algorithms and frameworks which detect and remove all PII from the ingested medical imaging data. The generated privacy law compliant and PII-compliant medical imaging data and PII metadata, which is PII-compliant by preferably being electronically stored without PII, provides for the health care provider and third party compliance with applicable privacy laws and federal regulations. In a preferred embodiment, the system and method ensures compliance with privacy laws and regulations governing the use of electronic health records. Preferably, the system and method removes PII from the medical data to promote the exchange of the data across third party health care providers. By removing any available PII, the system preferably improves quality of care by complying with the individual's right to privacy under current laws and regulations. The privacy law compliant medical data, without any PII, may be freely transmitted and exchanged among third parties for meaningful use, such as ensuring privacy and security of the individual's data, improving quality of care, reducing health care costs, eliminating waste, and increasing efficiency of healthcare providers at scale.

In a preferred embodiment of the system and method, medical data and electronic health records are ingested, PII is removed, and the data is outputted or re-written as new medical data, without PII and in strict compliance with privacy laws and regulations. The output medical data and electronic records may be accessed by third party analytics providers for the development of medical insights such as medication or drug interaction, or drug allergy checks, providing the state of current diagnosis across patient populations, improving patient outcome through diagnosis of disease states, comparing patient quality of care measures, streamlining clinical data or clinical trial research, or researching the safety and efficacy of new medical treatments, medications, or procedures.

In a preferred embodiment of the system and method, the medical data may be stripped of detectable personally identifiable information (PII) to build a large or massively scaled privacy law compliant data set. For example, a set of medical imaging and video data specific to a type of arthroscopic surgical procedure, may be ingested into the system and re-formatted for the removal of all PII. The system preferably outputs a new, re-written data set to build a massively scaled library of privacy law compliant, and PII-cleansed, library of medical imaging and video data of a patient population of arthroscopic surgery videos specific to a type of procedure. For example, the system may output and build a massive data set of knee surgery videos, without PII. The advantages of a scaled data set allow the mobilization and exchange of medical data and health care information across third party organizations for further data analytics and processing. Preferably, the advantages of a system may include the increased mobility and portability of medical data from a patient population, the improved efficiency and reduced cost of medical data storage, transmission and retrieval, and the development of patient outcome diagnosis and improvements of health care across patient populations.

In a preferred embodiment of the system and method, artificial intelligent powered models may be applied to the generated PII-compliant medical imaging data. A preferred embodiment may be the generation of a large data set of HIPPA (health insurance portability and accountability act) compliant medical imaging data for access, analysis and processing by third party data analytics firms for the development of anatomical recognition models, surgical metrics, patient outcome predictions, disease state diagnosis, medical payments, and billing data. For example, a large data set of endoscopic procedure videos may be made available to a third party analytics provider to mine for the development of anatomical recognition models specific to a type of medical procedure. In a preferred embodiment and use case, a third party analytics firm may build a large PII-compliant data set of knee surgery medical video data. The data may be used to develop anatomical recognition models to classify injury types and surgical procedural metric data. For a given surgical procedure, the data may be analyzed to generate patient outcome diagnosis, surgeon ratings, and medical billing data.

In a preferred embodiment of the system and method, the medical data may include patient specific text such as name, address, phone number, medical history, billing data, or account numbers. The system will preferably use AI-powered OCR (optical character recognition) models and frameworks to detect the presence of text-based personally identifiable information which may be linked to the patient or a specific individual. The text based PII may be present in medical imaging, video, patient medical records, or billing history files. The system preferably detects, tags, labels, or codes any instances of available text based PII. Thereafter, the PII is either removed, deleted, or redacted from the patient medical data and the system outputs a new re-written file, without PII, and the system additionally generates metadata describing the deleted PII. The PII-compliant medical data and records may be mined for doctor patient diagnosis patterns, medical billing efficiencies and auditing, or other medical record searching and statistical data development.

In a preferred embodiment of the system and method, medical data and medical imaging files may be formatted in the DICOM (digital imaging and communications in medicine) data format. The medical imaging data may include the patient's name, identification number, or other data which may be linked to the individual, embedded in the data or file format. The system and method may receive medical data in the DICOM format and thereafter apply AI-powered software and models to detect personally identifiable information in the DICOM formatted medical data. The PII is thereafter tagged, labeled, and otherwise marked for deletion and removal by the system. The system preferably processes the raw medical data on a frame-level basis, and removes any available DICOM data objects or PII which may be linked to any individual. The cleansed medical data is then re-written into new file formats and generates metadata describing the personally identifiable information and personal DICOM data objects that were removed from the raw data. The metadata may be checked to confirm the presence of certain known types of PII, whether the PII is being accurately detected by the system, and to confirm the amount of PII removed from the data.

In a preferred embodiment of the system and method, medical imaging and video data may be captured from a large amount of rotator cuff surgeries. Preferably the system receives and ingests a dataset of arthroscopic rotator cuff repair videos. The videos will typically show the arthroscopic repair of the rotator cuff by re-attaching the supraspinatus tendon to the bone using suture anchors. During the surgery, the captured video will show the insertion of the arthroscope into the shoulder through an incision or portal. The tissues, cartilage, bones, tendon, and ligaments surrounding the shoulder joint are typically inspected by the surgeon; saline is pumped into the joint to expand the area and facilitate inspection. Scar tissue, bone spurs, or damaged cartilage is usually shaved and removed from the joint to improve shoulder movement and biomechanics. To repair the rotator cuff, additional incisions or portals may be made around the joint to allow the insertion and placement of surgical instruments and tools. The supraspinatus tendon is preferably re-attached to the bone with sutures.

The system and method preferably applies AI-powered software to detect the presence of personally identifiable information (PII) in the dataset of rotator cuff repair videos. PII data may be present when the arthroscope is removed from the patient and inadvertently captures faces in the operating room. PII may additionally be present within the medical video file format, such as patient name, ID number, and also in any available audio data. The detected PII is deleted, removed or redacted by the system and the data set of repair videos is re-written in a cleansed format, which is compliance with health care privacy laws and regulations. Thereafter, the dataset may be analyzed, processed and mined for anatomical recognition, surgical analytical metrics, patient outcome diagnosis, medical billing purposes, and other secondary surgical data insights

In a preferred embodiment of the system and method, medical imaging and video data may be received and captured from a large data set of arthroscopic knee surgery videos. Preferably the system receives and ingests a dataset of arthroscopic knee surgery meniscus repair videos. The videos will typically show the arthroscopic repair of the knee joint. The video will show the arthroscopic video of the inside of the knee joint, captured with the insertion of a arthroscopic camera, and the repair and reattachment of the torn meniscus. Surgical tools are inserted into the incisions to repair the joint, remove scar tissue, and attach sutures. The system and method preferably applies AI-powered software to detect the presence of personally identifiable information (PII) in the dataset of knee surgery videos. PII data may be present when the arthroscope is removed from the patient and inadvertently captures the face of the patient, doctor, nurse or others in the operating room; additionally PII may be present within the medical video file format. The detected PII is deleted, removed or redacted by the system and the data set of knee repair videos is re-written in a privacy law compliant format. Thereafter, the dataset may be analyzed, processed and mined for secondary surgical data insights.

In a preferred embodiment of the system and method, a collection of endoscopic sinus surgery videos and medical data may be received and ingested onto a health care provider's computer and information technology systems. The endoscopic videos will typically show the images captured from the insertion of a tiny camera endoscope into the sinuses and examination of sinus tissues. Blockages, nasal polyps and scar tissue may be seen on the endoscope video images and are typically removed with surgical tools and instruments. The septum may be straightened with additional surgical procedures and tools on the captured video. The system and method preferably ingests the data sets of endoscopic sinus surgery onto the computer system or the health care provider's information technology systems. Artificial intelligence based software tools and models are applied to the endoscopic sinus surgery videos to examine, on a frame-level basis, for the detection and presence of personally identifiable information (PII), such as the patient's face, doctor or nurses faces, audible voices, patent name, medical history, or any health care information which may be linked to any individual. The system software-based tools thereafter label, tag, or code the detected PII for removal, deletion, or redaction. The dataset of endoscopic sinus surgery video are thereafter re-written without PII onto the health care provider's computer and information technology systems. Metadata describing the deleted PII is also provided to ensure that specific types of PII were indeed detected, and removed, and to give metrics regarding the removal of personally identifiable information. The privacy law compliant endoscopic sinus surgery video dataset may preferably be transmitted, uploaded, or downloaded to a secondary medical data storage network or information storage and retrieval system. The endoscopic surgery videos may preferably be normalized, stored, and organized in a hierarchical structure. Artificial intelligence based software tools and models may further process the video to ensure that all PII is in fact removed, and to confirm compliance with privacy laws and regulations. The PII-compliant endoscopic surgery video dataset may be transmitted to third parties for the generation of analytics, medical insights, anatomical recognition, surgical metrics, patient outcomes and diagnosis, and medical billing audit and payment analysis.

In a preferred embodiment of the presently described invention, a tangible computer readable medium comprising processor executable code or software is provided. The executable software code and tools may cause the CPU (central processing unit) or GPU (graphics processing unit) to perform certain functions, such as ingesting medical images, videos, and other data, detecting and removing personally identifiable information (PII), generating metadata, re-writing medical data for compliance with privacy laws and regulations, and applying artificial intelligence powered software and models to the data. The CPU or GPU may be integrated on the health care provider's information technology systems with available RAM or memory, logic controllers, communication and network devices, and information storage and retrieval systems.

1. A system for removing personally identifiable information from medical data comprising:
a data ingestion pipeline for receiving medical data from a health care provider;
artificial intelligence powered software tools and models for detecting personally identifiable information;
a software tool for removing, deleting, or redacting personally identifiable information;
a software tool for re-writing the medical data without any personally identifiable information (PII) and generating PII metadata;
a software tool for normalizing and storing the medical data in a hierarchical structure; and
a software tool for ensuring strict compliance with privacy laws and regulations; wherein, the personally identifiable information (PII) is any medical healthcare information which can be linked to any specific individual; wherein, the medical data is medical images, video or other electronic data from endoscopic cameras, arthroscopic surgeries, or other medical procedures or examinations; and wherein the system and software tools are interfaced with the healthcare provider computer and information technology systems.

2. The system of claim 1 for removing personally identifiable information from medical data, wherein the data ingestion pipeline provides a user interface and interactive experience, and wherein the received medical data provides an output identifying segments of personally identifiable information and functionality for user applied labels.

3. The system of claim 1 for removing personally identifiable information from medical data, wherein the software tools are executed in a HIPAA compliant cloud based computer system.

4. The system of claim 1 for removing personally identifiable information from medical data, wherein the medical data is used to build a massively scaled data set of privacy law compliant medical data for the improved exchange and analytics of healthcare data across patient populations.

5. The system of claim 1 for removing personally identifiable information from medical data, wherein the system outputs PII-cleansed medical data specific to a certain type of medical or surgical procedure and is used to develop medical insights for improving patient diagnosis and outcome.

6. The system of claim 1 for removing personally identifiable information from medical data, wherein the software tools are provided with a user interface for adapting the artificial intelligence powered PII detection models based on the generated PII metadata, to ensure strict compliance with privacy laws and reliable detection and removal of PII.

7. The system of claim 1 for removing personally identifiable information from medical data, wherein the system provides a user configurable PII detection and labeling interface for training the artificial intelligence powered tools for automated removal of PII at scale.

8. A system and method for removing personally identifiable information (PII) from medical data, comprising the steps of:
ingesting medical data from a healthcare provider onto a computer hardware and software system;
applying artificial intelligence powered software and models to detect personally identifiable information;
removing the detected personally identifiable information;
re-writing the medical data without personally identifiable information and generating metadata to describe the deleted PII; and
ensuring compliance with privacy laws and regulations.

9. The method of claim 8 for removing personally identifiable information (PII) from medical data, wherein the system provides a user interface for the manipulation of the ingested medical data and an output identifying segments of personally identifiable information and functionality for user applied PII-labels.

10. The method of claim 8 for removing personally identifiable information (PII) from medical data, wherein the steps for ingesting medical data, detecting and removing PII, and re-writing privacy law compliant medical data are executed in a HIPAA compliant cloud based computer system.

11. The method of claim 8 for removing personally identifiable information (PII) from medical data, wherein the medical data is outputted and used to build a massively scaled data set of privacy law compliant medical data for the improved exchange and analytics of healthcare data across patient populations.

12. The method of claim 8 for removing personally identifiable information (PII) from medical data, wherein the system and method outputs PII-cleansed medical data specific to a certain type of medical or surgical procedure and is used to develop medical insights for improving patient diagnosis and outcome.

13. The method of claim 8 for removing personally identifiable information (PII) from medical data, wherein the method provides for a user interface for adapting the artificial intelligence powered PII detection models based on the generated PII metadata, to ensure strict compliance with privacy laws and reliable detection and removal of PII.

14. The method of claim 8 for removing personally identifiable information (PII) from medical data, wherein the method provides for a user configurable PII detection and labeling interface for training the artificial intelligence powered software and models for automated removal of PII at scale.

15. A system and method for detecting and removing personally identifiable information (PII) from a patient population of medical data, comprising:
a user-interface for ingesting medical data and applying labels to PII;
an artificial intelligence powered software model which is trained by PII-labeled medical data for the further detection and labeling of PII across massive datasets representing a patient population;
a software tool for processing and removing PII across massive datasets of PII-labeled medical data;
a software tool for re-writing massive datasets of medical data without PII and generating PII metadata to ensure compliance with privacy laws and regulations; and
a software tool for providing access and exchange amongst third party data analytics for the development of medical insights in privacy law compliant medical data; wherein the system and method increases the mobility and exchange of medical data across health care providers; and wherein patient diagnosis and outcomes are iteratively improved as the system builds massively scaled labeled datasets of actionable intelligence for healthcare providers.

16. The system and method of claim 15, for detecting and removing personally identifiable information (PII) from a patient population of medical data, wherein the software tools are executed in a HIPAA compliant cloud based computer system.

17. The system and method of claim 15, for detecting and removing personally identifiable information (PII) from a patient population of medical data, wherein the system outputs PII-compliant medical data for a specific medical procedure for the development of highly-specialized and unique anatomical recognition models and disease state diagnosis methods.

18. The system and method of claim 15, for detecting and removing personally identifiable information (PII) from a patient population of medical data, wherein the software tools are provided with a user interface for adapting the artificial intelligence powered PII detection models based on the generated PII metadata, to ensure strict compliance with privacy laws and reliable detection and removal of PII.

19. The system and method of claim 15, for detecting and removing personally identifiable information (PII) from a patient population of medical data, wherein the software tools provide a user configurable PII detection and labeling interface for training the artificial intelligence powered tools for automated removal of PII at scale.

20. The system and method of claim 15, for detecting and removing personally identifiable information (PII) from a patient population of medical data, wherein the actionable intelligence facilitates the detection of anatomical features, such as bones, ligaments, and tendons, for the guidance of surgeons during medical procedures.
